(19) United States
(12) Patent Application Publication
(10) Pub. No.: US 2022/0227564 A1
(43) Pub. Date: Jul. 21, 2022

(54) OXYGEN ACTIVATED HEATER AND METHOD OF MANUFACTURING THE SAME

(71) Applicant: RBCE TECH, LLC, Cleveland, OH (US)

(72) Inventors: Christopher Pedicini, Franklin, TN (US); Darko Marquez, College Station, TX (US); Jeffrey Speich, Hockley, TX (US); John Beckerdite, College Station, TX (US)

(21) Appl. No.: 17/609,459

(22) PCT Filed: May 11, 2020
(86) PCT No.: PCT/US2020/032290

§ 371 Date: Nov. 8, 2021

Related U.S. Application Data
(60) Provisional application No. 62/846,049, filed on May 10, 2019.

(51) Int. Cl. B65D 81/34; F24V 30/00

(52) U.S. Cl. CPC B65D 81/3484; C09K 5/14; F24V 30/00

(57) ABSTRACT

A heating device having at least one package surrounding a hygroscopic salt and a heater mix having at least a metal reactant. The heating device may include a source of moisture positioned in the package, the source of moisture initially generating an atmosphere having nearly 100% relative humidity inside the package. The hygroscopic salt is spatially separated from the source of moisture within the at least one package and positioned so as to absorb moisture out of the atmosphere to form an electrolyte, and the hygroscopic salt is further positioned so that the electrolyte generated by the hygroscopic salt is in contact with the heater mix. Rather than have an internal source of moisture, the at least one package may be water vapor permeable to allow ambient moisture from the atmosphere to enter the interior of the package to combine with the hygroscopic salt to generate an electrolyte.

### RELATED APPLICATIONS

The present application claims the filing benefit of U.S. Provisional Patent Application No. 62/846,049 filed May 10, 2019—the contents of which are expressly incorporated herein by reference in their entirety.

### FIELD OF THE INVENTION

The present invention relates to oxygen activated heaters which use an exothermic reaction to generate heat and/or absorb oxygen.

### BACKGROUND OF THE INVENTION

Heaters which generate heat using an exothermic reaction based on the reaction of a metal reactant with oxygen to form a metal oxide are comprised of several key components. For example, these heaters include a heater material which may be in the form of a heater sheet or other format and includes a reactant material (such as a metal which is capable of reacting with oxygen, typically zinc or iron), carbon, and an optional binder (polytetrafluoroethylene (“PTFE”), for example). The heaters may further include an electrolyte which comes in contact with the reactant material, and serves as the medium for the exothermic reaction. The heater material may also include components to absorb the electrolyte (for example wood flour) or which serve to prevent unwanted clumping of the reactants (for example Vermiculite). The heater may further include a diffuser layer which rests on top of the heater material and beneath any outer packaging layer containing the air-access portion of the packaging layer. The diffuser is an air-flow-limiting membrane that can be used to manage the reaction rate (and thereby the heat released) during the metal oxide forming process. Finally, the heater includes an outer, oxygen-impermeable package which, when opened, exposes the heater reactant to the outside air to begin the heat generating reaction by allowing the reactant material to begin reacting with the oxygen in the air outside the package.

During manufacture of these heaters, premature activation of the heater material is of great concern. Once the electrolyte solution is introduced to the reactant material, the heater can be activated by any oxygen in the manufacturing environment. While steps can be taken to reduce exposure to oxygen, either by removing oxygen from the atmosphere or by reducing the time the activated reactant material is exposed to oxygen, premature activation prior to final sealing of the heater material and electrolyte in a sealed package results in unwanted waste of reactant material during manufacture. Depending upon the total amount of reactant material in a particular heater and the exposure time of the activated reactant material to oxygen in the manufacturing environment, as much as 5-10% of the weight of the reactant material may be used up before the heater is sealed in the outer package cutting off all atmospheric oxygen to stop the premature reaction. If there is any interruption during production, more heater material may be wasted in some heaters as sealing may be delayed for an extended period of time.

It would be advantageous to create a heater and method of manufacturing a heater which has a metal reactant which generates heat when exposed to oxygen using an exothermic reaction, while preventing the metal reactant from activating during the manufacture and packaging process.

### SUMMARY OF THE INVENTION

The present invention improves upon the known heaters and heater construction methods by providing a heater wherein the medium required for the reaction (for example, electrolyte) is generated within the oxygen-impermeable package after the heater is sealed. In order to prevent premature activation, rather than provide an electrolyte solution which mixes with the reactant material during manufacture prior to sealing the heater inside the oxygen-impermeable package, the present invention provides the necessary elements to create the electrolyte solution within the package over a period of time after the package is sealed, allowing for the heater to be completely sealed off from oxygen before any significant amount of electrolyte is created, activating the metal reactant. In order to further ensure that the premature activation does not occur during manufacture, the components for generating the electrolyte solution within the package (hygroscopic salt and a source of moisture) may be spatially separated from each other so that interaction between the electrolyte solution generating elements is delayed until after sealing the oxygen impermeable package.

In order to achieve this improvement, a package surrounding a heater material or heater mix (either a heater sheet, a dry heater powder, a heater substrate, or other form of a heater as described herein) and a hygroscopic salt which creates an electrolyte solution after absorbing water or water vapor is provided. A source of moisture or water is located either inside the package or absorbed from the atmosphere outside the package through the packaging, with the moisture or water being physically separated from the salt initially. Hygroscopic salts are chosen for their hygroscopic properties and the initially-dry salts will absorb water or water vapor from the local environment over time. This wetted salt then forms an electrolyte “in situ” to activate the metal reactant. By providing a heater material in contact with (or in some embodiments directly mixed with) a hygroscopic salt, and a separate source of moisture like water or water vapor, the present invention and method provides all of the elements to activate the metal reactant within the package, and provides the required elements in a manner such that the elements will mix after packaging to preserve the elements for actual use when needed. Once the elements are packaged and allowed to mix, an exothermic reaction will occur once the metal reactant inside the package is exposed to oxygen.

According to one aspect of the invention, a heating device is provided. The heating device includes a heater mix having a metal reactant, a hygroscopic salt, and at least one package surrounding the heater mix and hygroscopic salt. A source of moisture or water is positioned in the package, the source of moisture or water being capable of generating an initial atmosphere having 100% or nearly 100% relative humidity inside the package, and maintaining an elevated relative humidity within the package as the hygroscopic salt begins to absorb the moisture out of the internal atmosphere. For the purposes of this invention nearly 100% means over 90%. The hygroscopic salt is spatially separated from the source of moisture within the at least one package, and is positioned to absorb moisture out of the environment within the package to form an electrolyte. The hygroscopic salt is further positioned so that the electrolyte formed by the hygroscopic salt is in contact with the heater mix.

The heating device may optionally include a second package, the second package surrounding the heater mix and the hygroscopic salt and be positioned within the at least one package. The source of moisture or water within the at least one package may be positioned outside the second package in order to help spatially separate the source of moisture from the heater mix and the hygroscopic salt.

Regardless of whether or not a second package is used, the at least one package may be constructed from an oxygen impermeable material. The oxygen impermeable material may be one or more of a metal foil, a metallized polyethylene terephthalate film, or a metallized polypropylene film. Where a second package is used, the second package may be constructed from a water vapor permeable, but liquid impermeable, material.

The metal reactant within the heater mix may be one or more of Zinc, Iron, Aluminum, or Magnesium. The heater mix may further include a binder. The binder may be one or more of polytetrafluoroethylene or polyethylene glycol.

The hygroscopic salt may include one or more of Potassium Hydroxide (KOH), Lithium Chloride (LiCl), (hydrated) Calcium dichloride (CaCl₂), Sodium Bromide (NaBr), Sodium Chloride (NaCl), Potassium Bromide (KBr), and/or Potassium Chloride (KCl). The hygroscopic salt may be integrated with the heater mix and/or may be integrated with a carrier placed in contact with the heater mix, or carrying the heater mix, inside the at least one package.

The source of moisture within the at least one package may be one or more of water, a wetted absorbent material, a hydrogel, a saturated desiccant bag, water-based personal care gel or liquid, water-based air-care compound, or free water separated from the heater mix during assembly. The source of moisture may be a separate package or pouch of water, with the integrity of the separate package or pouch of water is compromised after the at least one package is sealed. The source of moisture may also be any source contributing to the local humidity within the environment sealed within the at least one package.

According to one aspect of the invention, a method of constructing a heating device is provided. The method includes the step of constructing a heater mix using a metal reactant and carbon, forming a package using an oxygen impermeable material, placing the heater mix inside the package, and placing a hygroscopic salt inside the package. A source of moisture is also placed inside the package such that the source of moisture is initially spatially separated from the hygroscopic salt. Once the heater mix, hygroscopic salt, and the source of moisture are placed inside the package, the package is sealed.

The moisture source generates an atmosphere having 100%, or nearly 100%, humidity in the package once the package is sealed and maintains an elevated relative humidity within the package once the hygroscopic salt begins absorbing the moisture from the atmosphere. The source of moisture within the at least one package may be one or more of water, a wetted absorbent material (for example a cellulosic or super absorbent polymer), a hydrogel, a saturated desiccant bag, water-based personal care gel or liquid, water-based air-care compound, or free water separated from the heater mix during assembly. The source of moisture may be a separate package or a pouch of water, with the integrity of the separate package or pouch of water being compromised after the at least one package is sealed. The source of moisture may also ambient humidity in the atmosphere sealed within the at least one package.

The method may further include forming a second package using a water vapor permeable, but liquid impermeable, material, placing the heater mix and hygroscopic salt inside the second package, sealing the second package and placing the second package inside the package prior to sealing the package.

The method may further include the step of integrating the hygroscopic salt with the heater mix prior to placing the heater mix in the package or the second package. The hygroscopic salt may instead be integrated with a carrier, and the carrier placed in contact with the heater mix in either the package or the second package. Both the hygroscopic salt and the heater mix may be integrated with a single carrier.

The method may further include the step of heating the package after sealing the package with the heater mix, the hygroscopic salt, and the source or moisture being sealed within the package.

According to one aspect of the invention, a heating device is provided. The heating device has a heater mix comprising a metal reactant and carbon, a hygroscopic salt, and at least one package surrounding the heater mix and the hygroscopic salt with the at least one package being oxygen impermeable and (optionally) water permeable. The hygroscopic salt absorbs ambient moisture from the atmosphere outside the package through the at least one package when the package is sealed to form an electrolyte, with the hygroscopic salt being positioned so that the electrolyte generated by the hygroscopic salt is in contact with the heater mix.

According to one aspect of the invention, a method of using a heating device is provided. The method includes opening an outer package having at least a heater mix comprising a metal reactant and carbon, a hygroscopic salt, and a source of moisture therein, wherein at least a portion of the source of moisture has been absorbed by the hygroscopic salt to form an electrolyte, introducing a second source of moisture inside the outer package, and re-sealing the outer package to allow the hygroscopic salt to absorb at least a portion of the second source of moisture to reform the electrolyte.

According to one aspect of the invention, a heating device having a heater mix having a metal reactant and carbon, and a dry salt during is provided. The heating device includes at least once package surrounding the heater mix and the dry salt, and a source of moisture. After being sealed in the at least one package the dry salt absorbs moisture and becomes the source of the electrolyte, with the electrolyte being positioned in the at least one package so that the electrolyte is in contact with the heater mix.

Other advantages and aspects of the present invention will become apparent upon reading the following description of the drawings and detailed description of the invention.

### BRIEF DESCRIPTION OF THE DRAWINGS

FIG. 1 shows an embodiment of the invention;

FIG. 2 shows a cross-section of the embodiment of FIG. 1 taken along the line A-A.

FIG. 3 shows an embodiment of the invention;

FIG. 4 shows a cross-section of the embodiment of FIG. 3 taken along the line B-B.

FIG. 5 shows an embodiment of the invention;

FIG. 6 shows a cross-section of the embodiment of FIG. 3 taken along the line C-C.

FIG. 7 shows an embodiment of the invention;

FIG. 8 shows a cross-section of the embodiment of FIG. 7 taken along the line D-D.

FIG. 9 shows an embodiment of the invention;

FIG. 10 shows a cross-section of the embodiment of FIG. 9 taken along the line E-E;

FIGS. 11A-11F show graphical representations of the exothermic reaction of embodiments of the invention;

FIGS. 12A-12F show graphical representations of the exothermic reaction of embodiments of the invention;

FIGS. 13A and 13B show graphical representations of hygroscopicities of various hygroscopic salts contemplated for use with the present invention;

FIGS. 14A-14F show graphical representations of the exothermic reaction of embodiments of the invention;

FIG. 15 shows a graphical representation of the exothermic reaction of embodiments of the invention; and

FIG. 16 shows a graphical representation of multiple reactions conducted with an embodiment of the invention.

### DESCRIPTION OF THE INVENTION

While the present invention is susceptible to embodiments in many different forms, there are described in detail herein, preferred embodiments of the invention with the understanding that the present disclosures are to be considered as exemplifications of the principles of the invention and are not intended to limit the broad aspects of the invention to the embodiments illustrated.

FIGS. 1-8 show multiple embodiments, and cross-sections of the various embodiments, of a heating device according to the present invention. In each embodiment shown in FIGS. 1-8, the heating device includes an outer package, a heater mix or substrate containing a metal reactant which reacts with oxygen to generate heat in an exothermic reaction, a hygroscopic salt, and a moisture or humidity source. In the various embodiments, each of the heater mix or substrate, the hygroscopic salt, and the moisture or humidity source are sealed within the outer package. Once sealed, the hygroscopic salt absorbs the moisture resulting from the source of humidity or moisture in order to form an electrolyte which activates the metal reactant so that the exothermic reaction can occur when the outer package is opened and the metal reactant is exposed to oxygen.

In each embodiment, regardless of placement, the use of hygroscopic salt in place of a liquid electrolyte makes use of salts that tend to absorb water from the humidity in the environment. These salts then at least partially self-dissolves and generate the electrolyte solution in the package after sealing, with the generated electrolyte solution eventually acting as the medium for the exothermic reaction. Insofar as the electrolyte is not generated, or at least predominantly not generated, until after the package is sealed, premature activation of the metal reactant in oxygen containing packaging environments is avoided insofar as no electrolyte, or very little electrolyte, exists to act as the medium to carry out the reaction in the presence of oxygen while the heater device is manufactured and packaged.

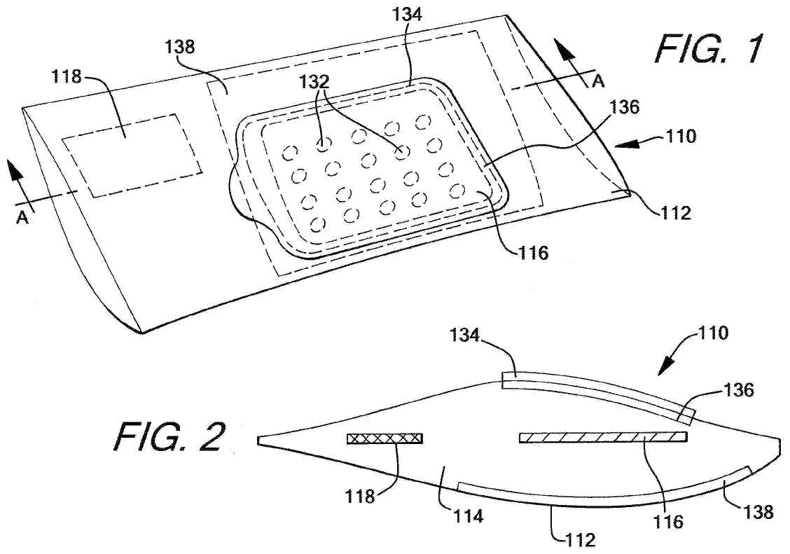

FIG. 1 and FIG. 2 which shows a cross-section along the line A-A in FIG. 1, show a first embodiment of the heating device of the present invention. As seen in FIGS. 1 and 2, heating device 110 includes an outer package 112 which has an interior area 114. Housed within interior area 114 is heater mix or substrate 116 and moisture or humidity source 118. Included in the heater mix or substrate is at least a metal reactant, and a hygroscopic salt is integrated with the heater mix or substrate. The metal reactant generates heat through an exothermic reaction when activated by an electrolyte and exposed to oxygen. The required electrolyte generated when the hygroscopic salt absorbs moisture and transforms at least partially into a solution, with the solution activating the heater mix when the heater mix is moistened or wetted by the electrolyte.

As seen in FIGS. 1 and 2, before sealing, humidity or moisture source 118 should be spatially separated or even isolated from the heater mix 116. By spatially separating the humidity or moisture source from the heater mix, and more specifically the hygroscopic salt, premature activation of the metal reactant can be avoided, as the required electrolyte to activate the metal reactant will not be immediately created and brought into contact with the metal reactant. However, by placing both the hygroscopic salt and the humidity or moisture source within the outer package and sealing the atmosphere, the moisture or humidity source will increase the relative humidity within the package and the hygroscopic salt over some period of time after sealing the outer package will absorb the required moisture or humidity within the package to dissolve and form the electrolyte solution required to activate the metal reactant.

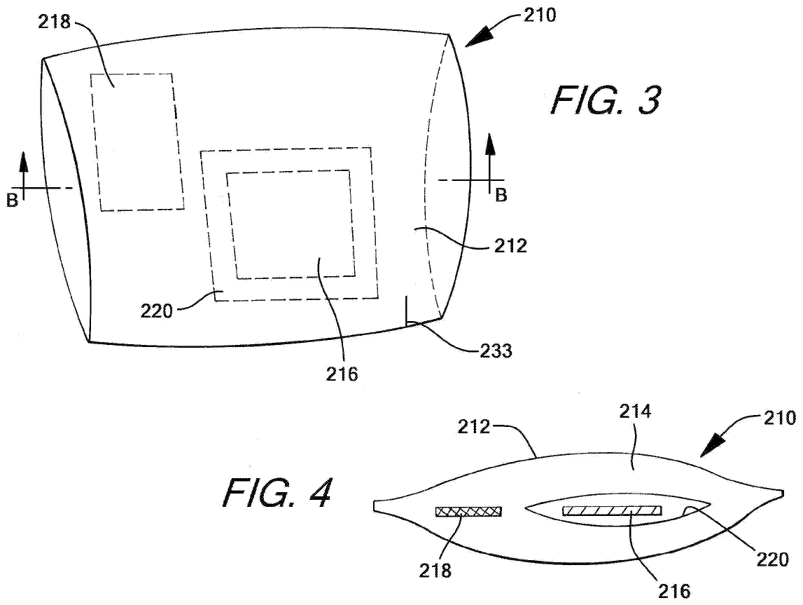

FIG. 3 and FIG. 4, which shows a cross-section along the line B-B in FIG. 3, show a second embodiment of the invention where additional steps are taken to prevent interaction between the source of moisture or humidity and the hygroscopic dry salt during the packaging process. As seen in FIG. 3 and FIG. 4, like heating device 110 shown in FIGS. 1 and 2, heating device 210 includes an outer package 212 which has an interior 214 which houses heater mix or substrate 216 and moisture or humidity source 218. However, before being housed in interior 214 of outer package 212, heater mix or substrate 216 is sealed in second package 220. Insofar as the heater mix or substrate is at least a metal reactant with a hygroscopic salt is integrated therewith, sealing the heater mix or substrate and integrated hygroscopic salt in the second package helps further prevent interaction between the hygroscopic salt and the moisture or humidity source during the manufacture process of the heating device.

Though not necessary to realize the benefits of utilizing a second package for the heater mix and hygroscopic salt during the manufacture process, it is particularly advantageous if the second package is made from a material that can allow for the release or transmission of water or water vapor. For example, second package 220 may be composed in part, or completely of, breathable film, which may be for example, cast-extruded embossed polyolefin film or spunbond polypropylene nonwoven material. The second package may comprise any film or material so long as some air access is provided within the package to allow the metal reactant to receive and react with oxygen when the heater is activated, and some humidity or vapor transmission is provided so that moisture can enter the package in order to wet or dissolve the dry salt and generate the electrolyte needed to activate the metal reactant.

In the first and second embodiments, the hygroscopic salt is integrated with the heater mix or heater substrate. In order to integrate the hygroscopic salt with the heater mix or substrate, the hygroscopic salt may be mixed with the heater reactant and any other elements during the formation of heater mix or substrate. Alternatively, the hygroscopic salt may be integrated by sprinkling it on top of the heater mix or sheet once formed. Regardless of how the hygroscopic salt is integrated with the heater mix or substrate, the heater mix or substrate may include one or more absorbent materials in order help hold and absorb moisture to interact with the hygroscopic salt to generate the electrolyte required to activate the metal reactant. Examples of absorbent materials which may be utilized include, but are not limited to, super absorbent polymers, wood pulp, vermiculite, or combinations thereof.

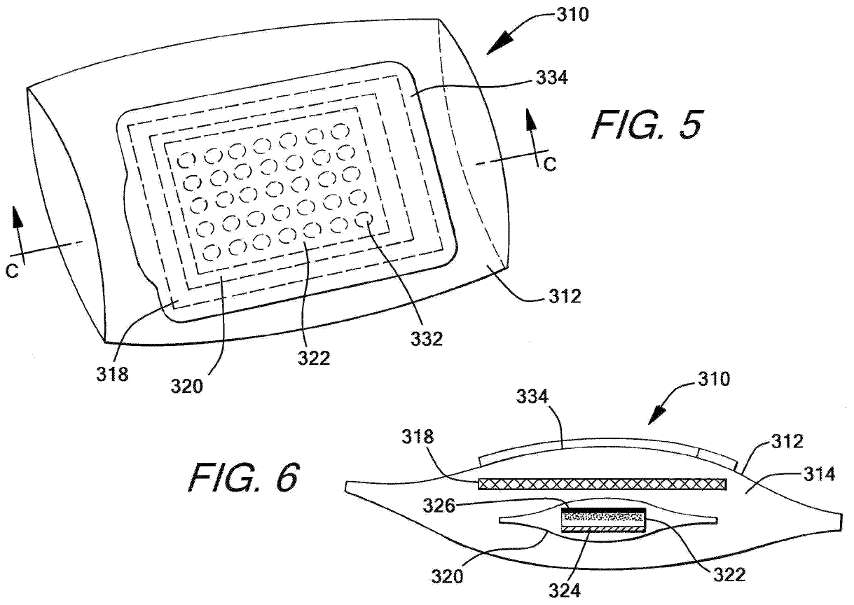

Of course, rather than integrating the hygroscopic salt directly with the heater mix or substrate, both the heater mix and hygroscopic salt may be integrated with a single carrier as seen in a third embodiment in FIG. 5 and FIG. 6 which is a cross-section of FIG. 5 taken along the line C-C. As seen in FIGS. 5 and 6, heating device 310 includes outer package 312 having an interior region 314 in which source of moisture or humidity 318 is housed. Also housed in region 314 is second package 320 in which carrier 322 is housed. Integrated with carrier 322 is a heater mix 324 and hygroscopic salt 326. Though shown in FIGS. 5 and 6 as including a second package, it is contemplated that a single carrier integrated hygroscopic salt and a heater mix may be utilized without a second package, for example, in place of heater mix or substrate 16 in FIGS. 1 and 2.

Integration of the heater mix and hygroscopic salt may be done in any manner of ways. For example, heater mix 324 may be coated on a first side of the carrier while hygroscopic salt 326 is integrated with the opposing side of the carrier. As the carrier and hygroscopic salt begin absorbing moisture, the electrolyte resulting from the at least partial wetting or dissolving of the hygroscopic salt may migrate through the carrier and engage and activate the metal reactant in the heater mix on the opposing side the carrier. Rather than, or in addition to being a coating, the carrier may be impregnated with one or more of the heater mix or hygroscopic salt.

In order to allow for integration and migration of electrolyte and/or heater mix, carrier 322 may be at least partially porous. For example, carrier 322 may be paper or other cellulosic material, an absorbent polymer, or any material capable of holding and or absorbing water. In addition, it is noted that the carrier 322 may also be used as the source of moisture within the outer package. Insofar as the hygroscopic salt may be formed on a top or bottom outside face of carrier 322, the carrier may be impregnated with water, for example, internally in a manner where the water is at least initially spatially separated from the layer of hygroscopic salt. So long as the carrier is sealed within an oxygen impermeable package prior to the salt and water combining forming electrolyte and before the electrolyte migrates to the heater mix on the opposing outer face or surface of the carrier, premature activation of the heater mix is avoided during the manufacture process.

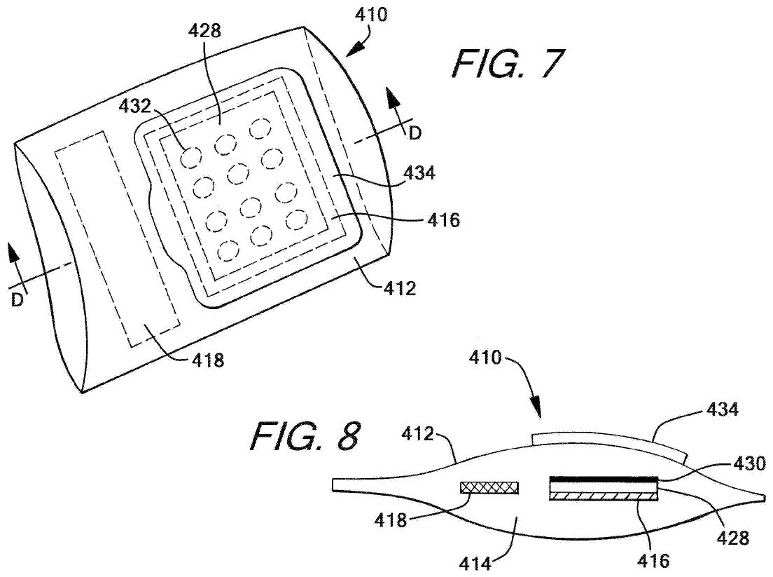

Rather than using a single carrier for both the hygroscopic salt and the heater mix, in a fourth embodiment of the invention shown in FIG. 7 and FIG. 8 which is a cross-section FIG. 7 taken through the line D-D, the hygroscopic salt may be integrated with a first carrier while the heater mix or substrate may be formed as an independent body or integrated with a second carrier. As seen in FIGS. 7 and 8, heating device 410 includes outer package 412 surrounding interior region 414 in which heater mix or substrate 416, humidity or moisture source 418, and carrier 428 are housed, with hygroscopic salt 430 being integrated with carrier 428. Carrier 428 is placed adjacent and in contact with heater mix on a separate second carrier or substrate 416 in order to ensure that electrolyte generated by the wetting or dissolution of hygroscopic salt 430 after outer package 412 is sealed. Though shown in FIGS. 7 and 8 without a second package, it should be understood the second package may be utilized with the carrier and integrated hygroscopic salt and the heater substrate or heater mix integrated with a second carrier being housed and sealed within the second package before being sealed as shown in FIGS. 3-6, for example.

In each embodiment of the present invention discussed thus far, each heating device has included a source of humidity or moisture housed within the outer package. The source of moisture or humidity within the at least one package may be one or more of water, a wetted absorbent material, a hydrogel, a saturated desiccant bag, water-based personal care gel or liquid, water-based air-care compound, or a pouch of water which is a separate package within the outer package, with the integrity of the pouch of water being compromised after the at least one package is sealed. Rather than be generated from a separate body or element within the outer package, the moisture or humidity source may be free water or humidity sourced from the heater mix or substrate during assembly of the heating device.

Regardless of the form of the source of moisture or humidity, the source of moisture or humidity will be of a determined amount based on a ratio of the metal reactant and hygroscopic salt amounts with a general approximation of 5%-30% of heater weight in water, for example, being provided within the heater. In practice, it is desirable to utilize an amount of water or moisture such that when the water or moisture is absorbed by the hygroscopic salt to the extent that the salt is at least partially dissolved, an electrolyte solution will be created having a weight in the range of 10-30 weight percent of the total heater mix or substrate weight. The effective electrolyte concentration may vary by salt type. Once the outer package is hermetically sealed, the water, moisture, or humidity source will begin hydrating the salt. As the salt absorbs the moisture out of the atmosphere in the package, and eventually at least partially dissolves as a result of the absorbed moisture, forming at least a partial electrolyte solution which acts as a medium for the exothermic reaction. The relative humidity within the package may drop during the absorption process, but will remain relatively elevated. Once the electrolyte solution is at least partially created within the sealed package, the metal reactant material will be ready for activation when the metal reactant material is exposed to oxygen in the air to start the exothermic reaction. In some embodiments/use environments, generating enough humidity internally for partial or short-term activation will suffice, with the remaining moisture being absorbed by the hygroscopic salt from the environment to continually generate and create electrolyte to act as the medium for carrying out the reaction.

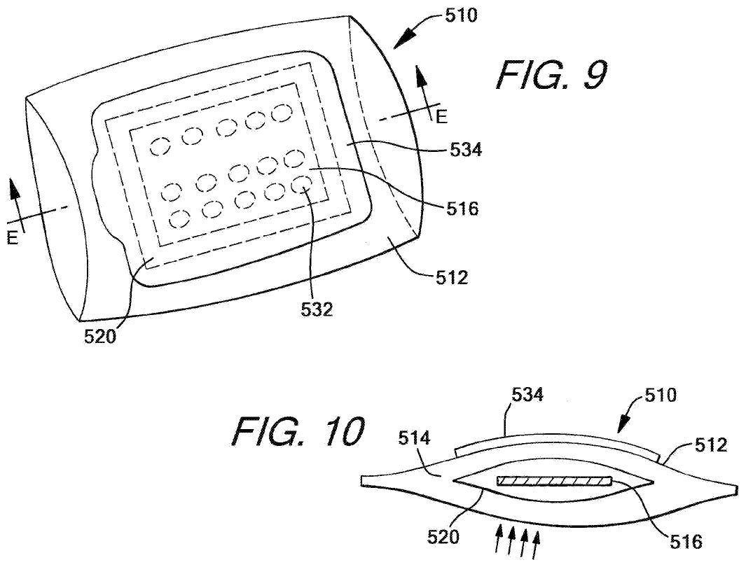

Rather than provide a source of moisture or humidity inside the outer package a fifth embodiment shown in FIG. 9 and FIG. 10 which is a cross-section of FIG. 9 taken along the line E-E, may not require any source of moisture or humidity be provided internally within the outer package, but rather all the moisture or humidity may be collected from the ambient environment outside the heating device.

As seen in FIGS. 9 and 10, heating device 510 includes outer package 512 which surrounds interior area 514 which houses a second package 520 which houses a heater substrate 516. As shown in FIGS. 9 and 10, heater substrate 516 includes at least a metal reactant with a hygroscopic salt integrated therewith with both housed within a second package, however it should be understood that the hygroscopic salt and heater mix may be housed directly in outer package 512 without a second package, similar to the heating device of FIGS. 1 and 2, and/or the hygroscopic salt may be formed on a single carrier with the heater mix similar to FIGS. 5 and 6, or on a carrier while the heater substrate is formed as a separate body or the heater mix is integrated with a second carrier similar to what is shown in FIGS. 7 and 8.

Without a source of moisture or humidity within package 512 as in the embodiments shown in FIGS. 1-8, the embodiment of FIGS. 9 and 10 uses ambient moisture or humidity from the atmosphere outside the heating device to interact with the hygroscopic salt and generate the required electrolyte to activate the metal reactant. As indicated by the arrows in FIG. 10, ambient moisture or humidity is permitted to pass through outer package 512 and eventually second package 520 when utilized to wet or dissolve the hygroscopic salt and generate the electrolyte.

Where a single package is used, the outer package material may be constructed from an oxygen impermeable material that has a high water vapor transmission rate to allow moisture to enter the package. Where two packages are utilized, outer package 512 may be constructed from an oxygen impermeable film, similar to outer packages 112, 212, 312, 412, with outer film 512 also having a high-water vapor transmission rate to ensure that a high level of moisture and humidity is generated in interior region 514. Second package 520 may then be made from an oxygen and water vapor permeable material in order to allow the hygroscopic salt to slowly generate the electrolyte and allowing oxygen to pass therethrough once oxygen is allowed to enter interior region 514 of the heating device. A material such as ethylene vinyl alcohol (“EVOH”) may be utilized, for example, which is substantially more permeable to water than oxygen.

In order to allow oxygen access to the interior of the outer package, the outer package in each embodiment may be provided with air access openings denoted by the reference numbers 132, 332, 432, 532, in each respective embodiment. In order to prevent premature access of oxygen into the interior of each outer package, a removable seal 134, 334, 434, 534, may be provided in each respective embodiment to seal the package. When a user desires to use the heating device, the seal can be partially or fully removed, allowing oxygen to pass through the openings to the interior of the outer package. Provided enough time has elapsed for the hygroscopic salt to partially or fully wet or dissolve, to generate the electrolyte solution required to carry out the heating reaction of the metal reactant, the exothermic reaction will begin once the metal reactant is exposed to the oxygen.

Alternatively, as seen in FIGS. 3 and 4, for example, rather than provide a removable seal and openings in the outer package, tear notch 233 may be provided in order to open outer package 212 and the heater mix to oxygen. A tear notch may be particularly useful, for example, in embodiments where a second, interior pouch is utilized, as the second pouch can be removed and utilized as the heater. Where a tear notch is used, it is contemplated that a zipper seal or the like may be utilized inside the first package so that the second package can be resealed therein for additional use.

In addition to the openings and seal, as seen in FIGS. 1 and 2, heating device 110 may include air diffuser 136 positioned between openings 132 and heater mix or substrate 116. The air diffuser may be constructed from an oxygen permeable, water impermeable material. The material of the air diffuser may be selected to achieve a desired oxygen transmission rate into the interior of the outer package in order to control the exothermic reaction and heat emitted by the heating device. These materials may be, for example, an apertured (for example needle or laser) polyolefin film or an inorganic-filled, porous polyolefin.

Additionally, the material of outer package 112 may be constructed from or include an insulating material, and/or a separate insulation material 138 may be provided inside or outside, along some or all of the outer package. Where a separate insulation material is used, the insulation material may be provided along a portion or the entire inner or outer surface of the package. The insulation material may be, for example, an “open” or “breathable” insulating material such as spunbond polypropylene nonwoven material or any other material which may be attached to the heater while remaining breathable to allow oxygen to pass therethrough.

Though only shown in FIGS. 1 and 2, it should be understood that an air diffuser and insulation material may be utilized with any of the embodiments of the heating device discussed herein. Likewise, a portion of the entirety of the outer package in embodiment may be constructed from, or include, an insulating material. In each embodiment, the air diffuser may be positioned as shown with respect to FIGS. 1 and 2, for example between the openings in the outer package and the heater mix or substrate. Insulation material may be provided by one or more of a separate material placed on the inner or outer surface of the outer package as shown in FIGS. 1 and 2, by making the outer package of an insulation material, and/or any carrier utilized in the interior of the outer package to carry the hygroscopic salt and/or heater mix or substrate may be made of an insulation material.

Likewise, the heater mix or substrate in each embodiment can take any form. For example, the heater mix may be a powder form held within the outer package or a second package, or may be a formed substrate, body, or sheet formed of the metal reactant and other any other materials.

The metal reactant in any of the heater substrates or mixes discussed with respect to each of the embodiments discussed herein may include one or more of Zinc (Zn), Iron (Fe), Aluminum (Al), Magnesium (Mg), or any other metal which oxidizes in the presence of oxygen. In addition to the metal reactant, the heater mixes or substrates may include one or more of carbon to act as a cathode for the exothermic reaction and help maintain structural integrity, a binder to hold the heater materials together (which is optional, and is generally PTFE or polyethylene glycol (“PEG”)), and/or absorbent materials to help absorb moisture to help wet or dissolve the hygroscopic salt to generate the required electrolyte solution (which are also optional and include but are not limited to super absorbent polymers, wood pulp, Vermiculite).

When the substrate is formed as a heater sheet, water may be added to the raw heater-mix materials described above, rolled out into a sheet and dried in an oven. The resultant sheet can be processed into rolls and handled without difficulty. Hence, using the traditional fabrication process salt is not added to the heater mix until the heater mix is dried because the heater mix is created in an open-air process and a combination of metal reactant/water/salt/carbon provide the necessary ingredients for the exothermic reaction to occur in the presence of air. Once such a heater sheet is created, however, hygroscopic salt may be sprinkled onto the dried sheet, or placed adjacent the dried sheet using a carrier or an air diffuser without substantially reduced concerns regarding premature activation of the heater.

Where a heater mix is used in the heating device, the heater mix may not be in the form of a physical sheet but is rather produced as a “dry mix.” Here the dry mix may contain the metal reactant, carbon, and (optionally) a binder such as PTFE or PEG. PEG in particular is highly useful in embodiments using a heater mix insofar as it has hygroscopic qualities of PEG allows for the absorption of additional moisture proximate the hygroscopic salt potentially allowing for longer heater operation without having to reintroduce a source of moisture to the heater to regenerate an electrolyte solution. In this instance the PTFE or PEG provides a minor amount of binder to the metal reactant/carbon mix to prevent the resultant powder from separating (the densities of zinc, for example, and carbon are quite different) back into its constituents.

Since the “dry mix” is not prepared with water, hygroscopic salt can be added directly to the dry mix, which simplifies production and potentially enhances any positive effect from the use of PEG as a binder. When using this embodiment of a heater mix or substrate or method where dry salt is added to the dry mix, there is no need for water removal, i.e. heating or drying, and salt can be directly incorporated into the heater mix itself, maximizing the effectiveness of the electrolyte solution generated once the package is sealed.

The hygroscopic salt in each embodiment discussed herein may be one or more of Potassium Hydroxide (KOH), Lithium Chloride (LiCl), (hydrated) Calcium dichloride (CaCl₂), Sodium Bromide (NaBr), Sodium Chloride (NaCl), Potassium Bromide (KBr), and/or Potassium Chloride (KCl). Hygroscopicity is an inherent property of salts, which can be defined as a chemical compound that results from the reaction of an acid with a base, with all or part of the hydrogen of the acid replaced by a cation, typically a metal cation. Some salts are inherently more hygroscopic than others and will absorb enough water (in vapor form) from the environment to self-dissolve, leading to the in-situ formation of a salt/electrolyte solution to activate the metal reactant. It is this salt solution that—when delivered to the heater mix or substrate with the metal reactant (e.g., a heater sheet)—can serve as the electrolyte to facilitate the activation of the metal reactant.

Insofar as different salts have different rates of absorbing water or moisture, the selection of which hygroscopic salt utilized within a heater can be chosen based on the desired speed with which the hygroscopic salt will wet or dissolve to form the required electrolyte and activate the heater mix or substrate within the sealed package. For example, in heating devices (or heaters) where fast activation within the package is desired/required, salts having a low relative humidity and a higher water absorption rate like MgCl₂, KOH, LiCl, CaCl₂, or NaOH may be utilized so that the electrolyte solution is generated in the package within a short time period after sealing. Where a slower rate of absorption is desired/required/possible within the sealed heater package, salts having a lower absorption rate like NaBr or NaCl, may be utilized, allowing for the heater material to become activate over a longer period of time within the sealed package. Using a salt having a slower absorption rate can further serve to prevent the formation of the electrolyte during the manufacturing and packaging process.

By the same token, the overall heat of the heater generated may be controlled by proper hygroscopic salt type and electrolyte concentration. Too high of an electrolyte concentration (i.e. not enough absorbed liquid) will result in the heater performing poorly, or not performing at all.

Exemplary heating devices or heaters manufactured in accordance with the embodiments discussed herein will now be discussed to show how variations of the hygroscopic salt, amount of moisture or humidity, and environmental factors may affect heater performance.

For example, table 1 reflects the difference in absorption rate and performance for a heater having a heater mix or substrate comprising zinc (Zn) as the metal reactant, carbon as the cathode for the reaction, a PTFE binder, and two variations of hygroscopic salt mixed into the heater mix—NaBr and MgCl₂. Each mix was placed in an environment having a 100% relative humidity (RH) for one week before activation of the heater was attempted.

TABLE 1

| | | Start | | | | Water | Resulting |
|---|---|---|---|---|---|---|---|
| | | Salt | Start | Final | % Weight | Pick-up | Solution |
| Group | Salt | Weight | Weight | Weight | Gain | (g) | Concentration % |
| C01 | NaBr | 1.0 | 11.2 | 12.4 | 117% | 1.17 | 46% |
| C02 | NaBr | 1.0 | 11.2 | 12.0 | 84% | 0.84 | 54% |
| C03 | NaBr | 1.0 | 11.3 | 12.1 | 82% | 0.82 | 55% |
| D01 | MgCl2 | 1.0 | 11.3 | 12.7 | 138% | 1.38 | 42% |
| D02 | MgCl2 | 1.0 | 11.2 | 12.8 | 156% | 1.56 | 39% |
| D03 | MgCl2 | 1.0 | 11.2 | 12.8 | 158% | 1.58 | 39% |
| E01 | NaBr | 3.0 | 13.7 | 14.2 | 18% | 0.53 | 85% |
| E02 | NaBr | 3.0 | 13.6 | 14.3 | 25% | 0.74 | 80% |
| E03 | NaBr | 3.0 | 13.5 | 14.4 | 29% | 0.86 | 78% |
| F01 | MgCl2 | 3.0 | 13.6 | 18.1 | 149% | 4.48 | 40% |
| F02 | MgCl2 | 3.0 | 13.5 | 17.4 | 130% | 3.90 | 43% |
| F03 | MgCl2 | 3.0 | 13.6 | 17.5 | 129% | 3.88 | 44% |

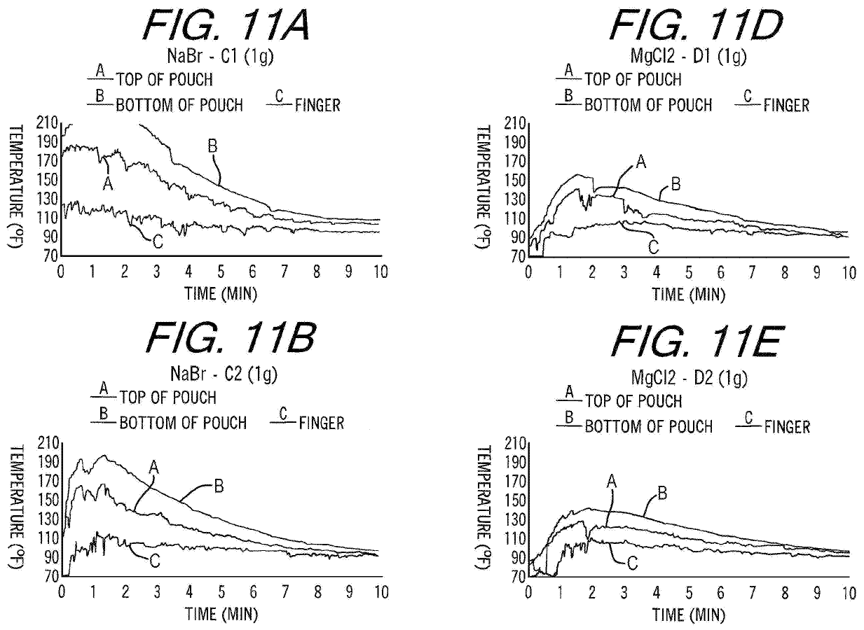

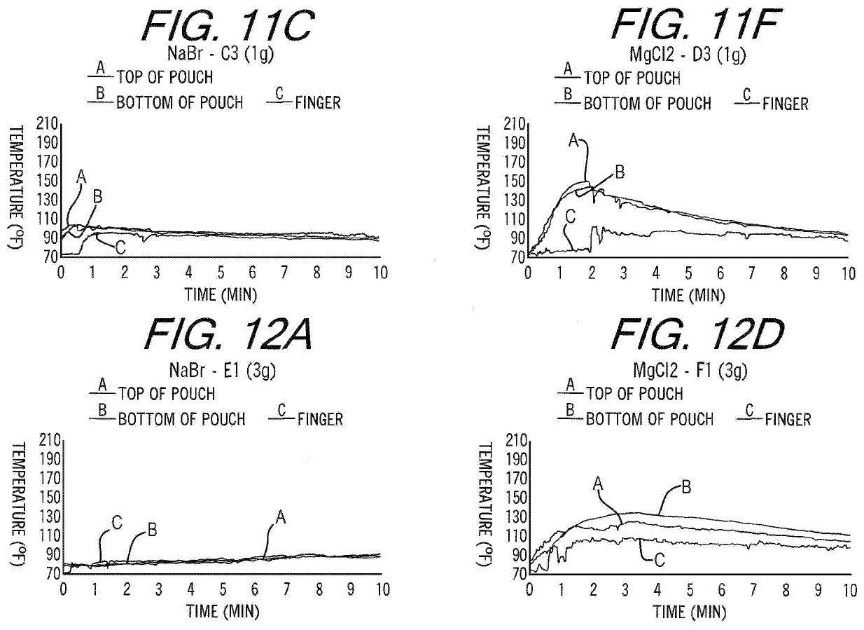

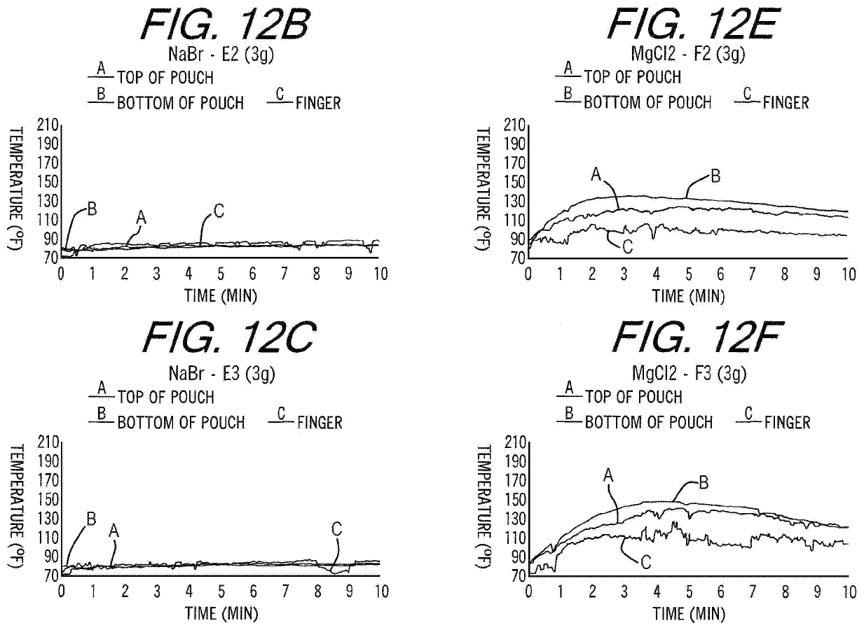

As seen in table 1, the heater mixes which included 1 g or 3 g of MgCl₂ absorb roughly 1.5×-8.5× the amount of water a similar amount of NaBr absorbs. The increased water absorption of the MgCl₂ resulted in a more dilute electrolyte solution. The resulting heaters performed as shown in FIGS. 11A-F and 12A-F.

As can be seen in FIGS. 11A-F and 12A-F, heaters which comprise MgCl₂ result in much more stable, predictable, and extended heating profiles measured at each of the top of the pouch or outer package (A), the bottom of the pouch outer package (B), and at a finger (C) than did the heaters comprising NaBr. The heaters which reach the highest immediate temperature, however, for a quick high temperature burst of heat, are heaters which comprise 1 g of NaBr as it resulted in an electrolyte which was reasonably concentrated yet prevalent enough to facilitate the reaction. The heaters containing 3 g of NaBr were not yet ready for performance after one week in the 100% humidity environment, as the NaBr failed to absorb enough water over the weeklong period to achieve a workable electrolyte within the package.

Of note, the amount of MgCl₂ does not have a significant effect on the overall temperature of the heater, the amount of MgCl₂ did, however, affect the amount of time required for the heater to reach the peak temperature, and the duration the heater remained active at or near the peak temperature. As seen in the heaters containing 1 g of MgCl₂, for example, the heaters quickly reached a peak temperature around 150° F., but quickly dissipated from there due to the ease with which the metal reactant (Zn) could access oxygen from the smaller amount of electrolyte precluding the flow of oxygen, and the rapid depletion of smaller amount of electrolyte solution through the evaporation of the water absorbed by the hygroscopic salt. The heaters comprising 3 g of MgCl₂ resulted in heaters which reached the same peak temperature around 150° F., albeit at a slightly slower rate, and maintained that peak temperature for a much longer period of time. The slower heating rate and extended activity being the result of the larger amount of electrolyte lasts longer before evaporation and which may prevent some of the metal reactant (Zn) from accessing oxygen immediately.

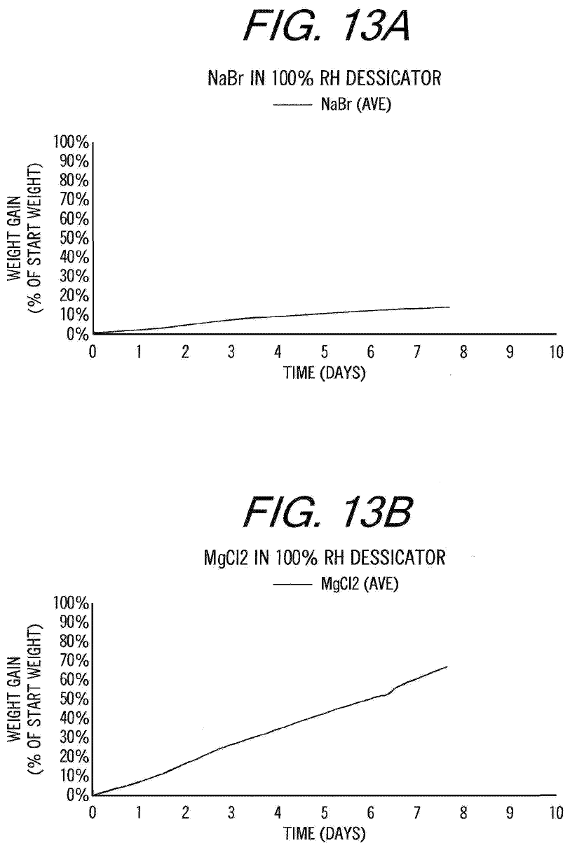

The absorption rates of the utilized salts—NaBr and MgCl₂ outside the heater mix provide further support for the absorption rates and performance of the heaters above, as seen in FIGS. 13A and 13B. As seen in FIGS. 13A and 13B, MgCl₂ is more hygroscopic by nature than NaBr, allowing the MgCl₂ to absorb water a faster rate. Use of the more hygroscopic salt will result in the metal reactant becoming active faster to allow for the heater to be utilized faster than a heater with NaBr. FIGS. 13A and 13B show heater device performance at a top (A), bottom (B), and finger (C) of the package.

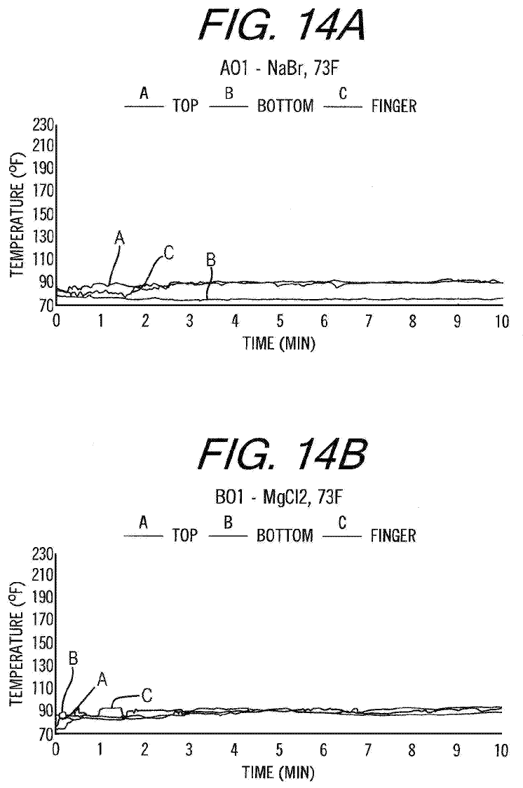

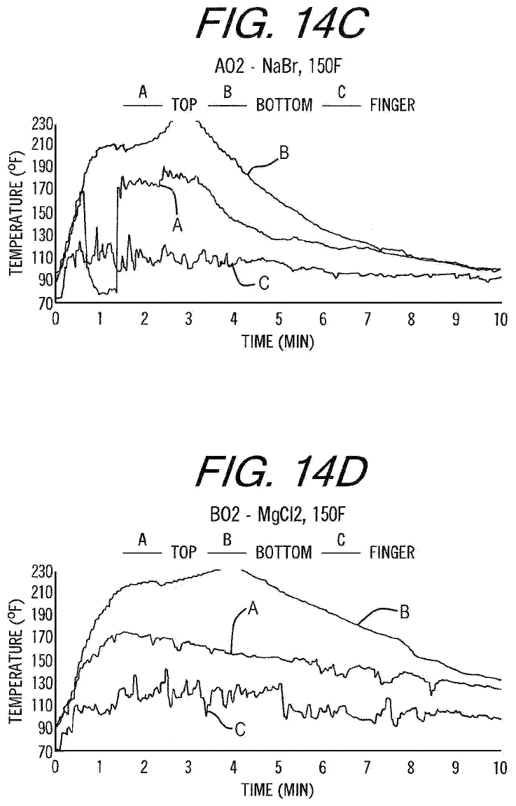

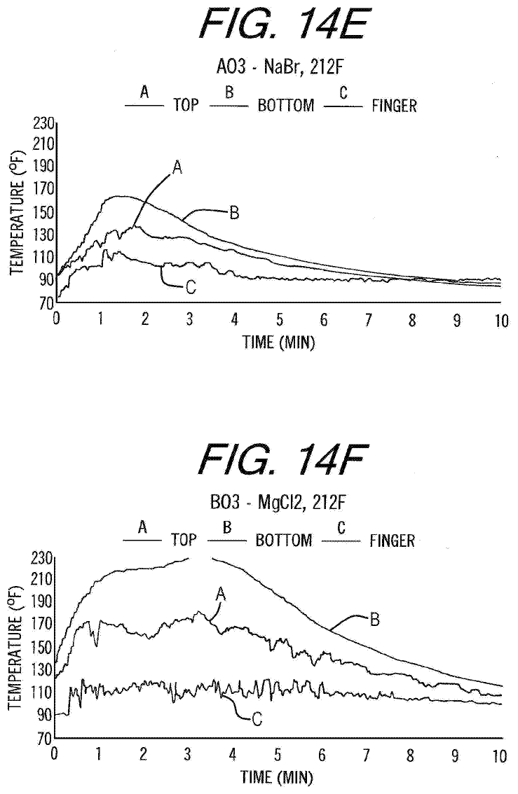

Rather than merely rely on the hygroscopic properties of a particular chosen salt within a heater, in order to achieve faster activation of the metal reactant within the sealed package, the storage or at least the initial storage, of the heaters may be controlled in a manner which promotes the absorption of moisture by the salt. For example, as seen in FIGS. 14A-14F storing the sealed heater in a pouch with water (2 g in each case shown below) for six (6) days at higher temperatures may result in faster activation of the heater in the sealed package. In each of the heater embodiments shown in FIGS. 14A-14F, each heating device or heater again contains a heater mix having Zinc, Carbon, PTFE and either MgCl₂ or NaBr.

As seen in FIGS. 14A and 14B, storing the heating devices and water within the sealed pouches at room temperature results in poor heater performance when the heater was activated in a short time after sealing, i.e. six days, as very little water evaporated and was absorbed by either salt. At the higher temperatures shown in FIGS. 14C-14F, the water within the package evaporates much faster when stored at the higher temperature, allowing more of the moisture to evaporate, enter the heater package, and be more easily absorbed by the salt to generate the electrolyte solution. By heating the heating device and using water as the source of moisture or humidity, the heater is allowed to become active faster as the heating of the heating device evaporates the water significantly speeds up the electrolyte creation process within the heater.

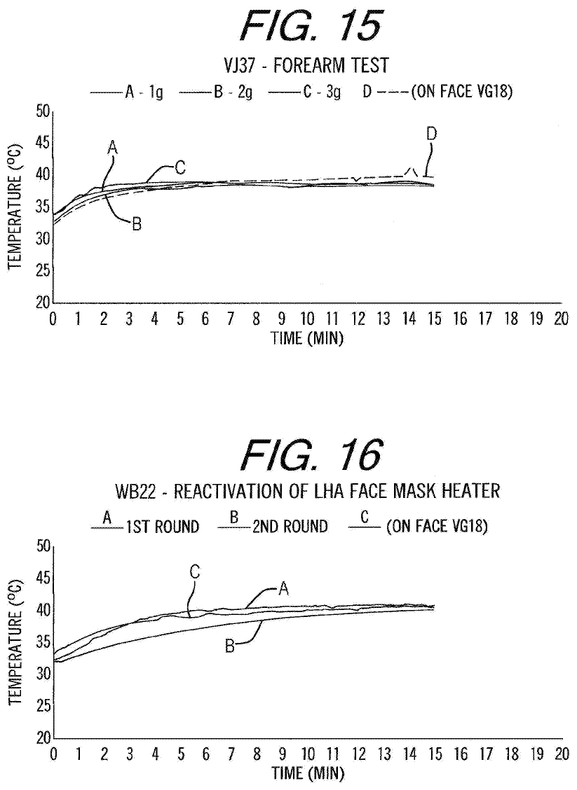

While the type of hygroscopic salt and altering the storage environment of the heating device may result in different heating profiles/characteristics of the heating device, the amount of water which is provided within the package does not affect heater performance so long as the amount of water does not overwhelm the heater and prevent exposure of some or all of the metal reactant to oxygen, thereby inhibiting the exothermic reaction. As seen in FIG. 15 and Table 2 below, whether 1 g (A), 2 g (B), or 3 g (C) of water is provided within a heater, and the heater is stored for a period of one week, the heating characteristics (both the time to reach the peak temperature and longevity of the heater) are not substantially affected. Each of the heating devices tested in groups A, B, C in FIG. 15 and Table 2 includes Zn, Carbon, PTFE, and 2 g of NaBr infused on a carrier and being stored in a heated environment for one week before activation.

TABLE 2
Standard Deviation (° C.)

| Groups | A | B | C |
|---|---|---|---|
| Average | 1 | 3 | 1 |
| Max | 1 | 3 | 2 |

Since the environment within the sealed outer package achieves a RH of 100% initially regardless of the amount of water which is placed into the pouch, over a one week period, and maintains an elevated relative humidity within the package as the salt begins to absorb the moisture, the same amount of moisture is absorbed by the salt in each case, resulting in a heater that performs in a similar manner, regardless of the amount of water which is provided.

An additional means by which the exothermic reaction can be controlled is by controlling the breathability of the outer package. For example, a more open or porous material results in a heater which heats faster, while a less open or porous outer package reduces and/or inhibits the ability of the heater to heat.

Utilizing hygroscopic salt has a further advantage over known heating devices in that it allows for a single heating device to be used multiple times. In present heating devices, after the initial exothermic reaction has taken place, the electrolyte typically evaporates to below a functional level before all of the metal reactant in the heating device is consumed. If a humidity source is reintroduced into the “outer package,” the re-saturated salt within or adjacent the heater substrate or sheet or heater mix can reactivate the metal reactant remaining within the heating device. Depending on the amount of the metal reactant consumed during each usage, utilizing an internal hygroscopic salt to generate the required electrolyte solution may allow for a heating device to be used multiple times rather than just once as with conventional heaters as any remaining metal reactant can continue to react with oxygen after the initial generated electrolyte in the heater is utilized. By reintroducing more liquid to the heater to re-saturate the salt—either directly by providing water or moisture into the package or ambiently through the atmosphere—the salt can re-saturate and generate new electrolyte to carry a second or subsequent reaction. This humidity (or water) reintroduction is particularly useful when combined with a re-sealable package. Using the method and heater of the present invention, a humidity source can be reintroduced into the heater package, the package re-sealed, and the moisture re-saturating the hygroscopic salt to regenerate electrolyte for the reaction. When the package is re-opened, and the activated metal reactant re-exposed to oxygen, a second round of exothermic reaction occurs. Alternatively, when the second use occurs in moist environments, like for example within a humid environment in a food package or in an environment where the ambient air is sufficiently humid, the moisture in the ambient local atmosphere may be used to either generate more electrolyte within the heater (by re-dissolving at least a portion of the salt), or to extend the time the heater is active as the ambient moisture may allow for the salt to continue to absorb water during use so that electrolyte solution is continually generated. Use of a low Oxygen Transmission Rate (“OTR”), high Water Vapor Transmission Rate (“WVTR”) film will facilitate activation using moisture in the ambient air or environment.

For example, FIG. 16 shows heater performance for a heater comprising Zn, Carbon, PTFE, and 2 g of NaBr infused on a carrier and being stored for one week before activation after being integrated into a therapeutic facemask for two uses of the heater.

As seen in FIG. 16, the heating device was able to be activated a first time (A) to generate heat on a user's face (C)—after being provided with 3 g of water to generate the electrolyte solution with the NaBr—and was able reactivate after being provided with 3 g of water to regenerate the electrolyte solution (B). While the second activation may take a longer period of time to reach the peak temperature of approximate 40° C., the heater is able to achieve nearly the same peak temperature over time during the second usage. Reactivation was accomplished by adding 3 more grams of water as the humidity source, the heater mix or substrate was placed back into the outer package, and the outer package was resealed. After three days the package was re-opened and re-used.

Regardless of heater form, an external source of water may be utilized for electrolyte formation. For example, when used as an oxygen scavenger within an opened wine bottle, the heater “package” is in the form of a bottle stopper with the air access portion being adjacent to the air space at the top of the wine bottle. Since this air is at approximately 100% relative humidity, the water in the air space with migrate into the salt and activate the heater. In this case, oxygen can be scavenged at a slow rate. Recall the zinc-oxygen (or other metal reactant-oxygen) reaction liberates heat but also consumes oxygen, creating an oxygen removal system that can address needs in the packaging industry where an inert or oxygen-free environment is desired (e.g., food packaging).

Additionally, a package that has low permeability to oxygen but has high permeability with respect to water may be utilized. Materials which may be utilized for such a package include but are not limited to EVOH and polyvinyl alcohol (PVOH). In this instance, water could slowly permeate from the outside environment, through the semi-permeable barrier, and into the salt in the interior of the heater. The salt will attract water through the packaging with the heater not becoming activated since the package has low oxygen permeability. Such is an alternative method of manufacture wherein heaters can be manufactured without any moisture or water source required, further limiting the possibility of premature activation. Once the heaters are sealed within the oxygen impermeable packages, in order to insure activation upon opening of the package, the packages may be placed within a moist or humid atmosphere in order to generate the necessary electrolyte solution prior to use. By placing no water or moisture source within the oxygen impermeable package, the possibility of accidently saturating the hygroscopic salt and activating the heater during manufacture is essentially reduced to zero prior to the package being sealed.

End-use applications for this technology could include packaging applications that need to exclude air (e.g., foodstuffs), negative pressure wound care (the atmosphere being 20% oxygen and having a natural local humidity of approximately 100%) where by absorbing the oxygen a reduced pressure is realized, thereby drawing out wound fluids. The heaters discussed herein may also be usable as heating elements to heat thermoformable materials, such as splints. Additionally, warming of a body part for comfort or to deliver therapeutic materials to the skin is a very important application for us, especially in the area of eye or face masks.

While in the foregoing there has been set forth preferred embodiments of the invention, it is to be understood that the present invention may be embodied in other specific forms without departing from the spirit or central characteristics thereof. The present embodiments, therefore, are to be considered in all respects as illustrative and not restrictive, and the invention is not to be limited to the details given herein. While specific embodiments have been illustrated and described, numerous modifications come to mind without significantly departing from the characteristics of the invention and the scope of protection is only limited by the scope of the accompanying claims.

What is claimed is:

1. A heating device comprising:
a heater mix comprising a metal reactant;
a hygroscopic salt;
at least one package surrounding the heater mix and the hygroscopic salt; and
a source of moisture positioned in the package, the source of moisture generating an atmosphere having nearly 100% relative humidity inside the package,
wherein the hygroscopic salt is spatially separated from the source of moisture within the at least one package and positioned so as to absorb moisture out of the atmosphere to form an electrolyte, wherein the hygroscopic salt is further positioned so that the electrolyte generated by the hygroscopic salt is in contact with the heater mix.

2. The heating device of claim 1 further comprising a second package, the second package surrounding the heater mix and the hygroscopic salt and being positioned within the at least one package, the source of moisture within the at least one package being positioned outside the second package.

3. The heating device of claim 1, wherein the at least one package is constructed from an oxygen impermeable material.

4. The heating device of claim 2, wherein the second package is constructed from a water vapor permeable, but liquid impermeable, material.

5. The heating device of claim 1, wherein the metal reactant comprises one or more of Zinc, Iron, Aluminium, or Magnesium.

6. The heating device of claim 1, wherein the heater mix includes a binder.

7. The heating device of claim 6 wherein the binder is one or more of polytetrafluoroethylene or polyethylene glycol.

8. The heating device of claim 1, wherein the hygroscopic salt includes one or more of Potassium Hydroxide (KOH), Lithium Chloride (LiCl), (hydrated) Calcium dichloride (CaCl₂), Sodium Bromide (NaBr), Sodium Chloride (NaCl), Potassium Bromide (KBr), and/or Potassium Chloride (KCl).

9. The heating device of claim 1, wherein the hygroscopic salt is integrated with the heater mix.

10. The heating device of claim 1, wherein the hygroscopic salt is integrated with a carrier, the carrier being placed in contact with the heater mix inside the at least one package.

11. The heating device of claim 3, wherein the oxygen impermeable material is a metal foil, a metallized polyethylene terephthalate film, or a metallized polypropylene film.

12. The heating device of claim 1, wherein the source of moisture one or more from the group consisting of wetted absorbent material, hydrogel, a saturated desiccant bag, water-based personal care gel or liquid, water-based air-care compound, or free water separated from the heater mix during assembly.

13. The heating device of claim 1, wherein the source of moisture is a pouch of water, wherein the integrity of the pouch of water is compromised after the at least one package is sealed.

14. The heating device of claim 1, wherein the source of moisture is ambient humidity in the atmosphere sealed within the at least one package.

15. A method of constructing a heating device, the method comprising the steps of:
constructing a heater mix using a metal reactant and carbon;
forming a package using an oxygen impermeable material;
placing the heater mix inside the package;
placing a hygroscopic salt inside the package;
placing a source of moisture inside the package, the source of moisture spatially separated from the hygroscopic salt; and
sealing the package.

16. The method of claim 15 wherein the source of moisture generates an atmosphere having 100% humidity inside the package once the package is sealed.

17. The method of claim 15 further comprising the steps of:
forming a second package using a liquid impermeable material;
placing the heater mix and hygroscopic salt inside the second package;
sealing the second package; and
placing the second package inside the package prior to sealing the package.

18. The method of claim 15, further comprising the step of integrating the hygroscopic salt with the heater mix prior to placing the heater mix in the package or the second package.

19. The method of any one of claim 15, further comprising the step of integrating the hygroscopic salt with a carrier, and placing the carrier in contact with the heater mix in either the package or the second package.

20. The method of claim 15, further comprising the step of heating the package after sealing the package with the heater mix, the hygroscopic salt, and the source or moisture being sealed within the package.

21. A heating device comprising:
a heater mix comprising a metal reactant;
a hygroscopic salt;
at least one package surrounding the heater mix and the hygroscopic salt, the at least one package being water permeable but oxygen impermeable,
wherein the hygroscopic salt absorbs ambient moisture through the at least one package when the package is sealed to form an electrolyte, wherein the hygroscopic salt is further positioned so that the electrolyte generated by the hygroscopic salt is in contact with the heater mix.

22. A method of using a heating device comprising the steps of:
opening an outer package having at least a heater mix comprising a metal reactant and carbon, a hygroscopic salt, and a source of moisture therein, wherein at least a portion of the source of moisture has been absorbed by the hygroscopic salt to form an electrolyte;
introducing a second source of moisture inside the outer package; and
re-sealing the outer package to allow the hygroscopic salt to absorb at least a portion of the second source of moisture to reform the electrolyte.

23. A heating device comprising:
a heater mix comprising a metal reactant;
an electrolyte, the electrolyte being dry during a manufacturing process;
at least one package surrounding the heater mix and the hygroscopic salt;
a source of moisture;
wherein the electrolyte absorbs the moisture after being sealed in the at least one package make the electrolyte wet, wherein the electrolyte is further positioned so that the electrolyte is in contact with the heater mix when it becomes wet.

24.-26. (canceled)
